(19) United States
(12) Patent Application Publication
(10) Pub. No.: US 2022/0346865 A1
(43) Pub. Date: Nov. 3, 2022

(54) MEDICAL PUNCHING/CAUTERIZING DEVICE

(71) Applicants: Yong Chul AHN, Seongnam-si, Gyeonggi-do (KR); Sang Koo JEON, Namyangju-si, Gyeonggi-do (KR); Sang Soo LEE, Seoul (KR)

(72) Inventors: Yong Chul AHN, Seongnam-si, Gyeonggi-do (KR); Sang Koo JEON, Namyangju-si, Gyeonggi-do (KR); Sang Soo LEE, Seoul (KR)

(21) Appl. No.: 17/263,223

(22) PCT Filed: Jul. 26, 2019
(86) PCT No.: PCT/KR2019/009349

§ 371 Date: Jan. 26, 2021

(30) Foreign Application Priority Data
Jul. 26, 2018 (KR) 10-2018-0087205

(51) Int. Cl. A61B 18/14

(52) U.S. Cl. CPC A61B 18/1477; A61B 2018/00595

(57) ABSTRACT

A medical punching/cauterizing device includes a handle having a body, a cap detachably coupled to a front portion of the body, a connection terminal coupled to the body and electrically connected to a power supply means, a conductor provided in the front portion of the body and having one end electrically connected to the connection terminal, and a guide wire provided on a front portion of the handle and having one end electrically connected to the conductor.

### CROSS-REFERENCE TO RELATED APPLICATION

This application is a U.S. national phase application of PCT International Application PCT/KR2019/009349, filed Jul. 26, 2019, which claims priority to Korean Patent Application No. 10-2018-0087205, filed Jul. 26, 2018, the contents of which are incorporated herein by reference in their entirety.

### TECHNICAL FIELD

An exemplary embodiment of the present invention relates to a medical punching/cauterizing device capable of being applied to endoscopic ultrasound-guided drainage or the like.

### BACKGROUND ART

Biliary and pancreatic diseases (bile duct cancer, gallbladder cancer, pancreatic cancer, pancreatitis, etc.) are increasing exponentially due to various causes such as population aging, dietary changes, and obesity, and endoscopic ultrasound-guided drainage is used as a method of treating these diseases.

The endoscopic ultrasound-guided drainage is much widely carried out worldwide in the gastrointestinal field. The endoscopic ultrasound-guided drainage is being performed on various diseases that could not be treated or needed to be subjected to surgery in the related art, and patient's satisfaction with the endoscopic ultrasound-guided drainage is also increasing. For example, in the field where the endoscopic ultrasound-guided drainage is currently used, there are performed drainage of pseudocyst which is a complication associated with pancreatitis, intrahepatic or extrahepatic bile duct drainage, gallbladder drainage, and drainage of fluid retention resulting from postoperative complications, and the field of application of the endoscopic ultrasound-guided drainage gradually expands worldwide.

However, the endoscopic ultrasound-guided drainage is one of the most difficult procedures among gastrointestinal procedures and has a disadvantage in that the procedure is comparatively complicated. The method commonly used for various procedures is as follows.

(1) A site required to be subjected to drainage is detected by endoscopic ultrasound. (2) A 19G puncture needle is inserted into a biopsy channel of endoscopic ultrasound to puncture the site required to be subjected to the drainage in the stomach or duodenum. (3) A guide wire is inserted through an interior of the puncture needle. (4) An electrocautery is inserted along the guide wire to expand a tract between the stomach or duodenum and the site to be subjected to the drainage. (5) The electrocautery is withdrawn and a stent is inserted along the guide wire.

During this process, it is very important to insert the electrocautery along the guide wire to expand the tract, remove the electrocautery, and then insert the stent along the guide wire. This is because there is a high risk that bile or a contaminated liquid will flow into the abdominal cavity once the tract is expanded. Therefore, the above-mentioned process needs to be quickly performed, but the above-mentioned process is the most difficult process during the actual procedure, and complications often occur in this process.

Further, the procedure time is important because the endoscopic ultrasound-guided drainage is performed as endoscopy under conscious sedation, like sleep endoscopy. This is because the increase in procedure time causes pain to a patient and also increases a risk of sleep-related complications. Therefore, it is very important to implement a device and a method which are capable of shortening the procedure time.

The reference may be made to Korean Patent Application Laid-Open No. 10-2018-0024963 as a relevant document in the related art.

### SUMMARY OF THE INVENTION

An exemplary embodiment of the present invention provides a medical punching/cauterizing device which is a single modularized device capable of safely performing endoscopic ultrasound-guided drainage. Another exemplary embodiment of the present invention provides a medical punching/cauterizing device capable of shortening the procedure time and reducing the number of procedures.

An exemplary embodiment of the present invention may provide a medical punching/cauterizing device including: a handle having a body, and a cap detachably coupled to a front portion of the body; a connection terminal coupled to the body and electrically connected to a power supply means; a conductor provided in the front portion of the body and having one end electrically connected to the connection terminal; and a guide wire provided on a front portion of the handle and having one end electrically connected to the conductor.

The cap may have a first through hole into which the guide wire is inserted, a first guide surface may be formed on a front portion of the cap and disposed around the first through hole, and a diameter of the first guide surface may decrease toward a rear portion of the cap.

A first fastening part may be provided on the front portion of the body, a first screw thread may be formed on an outer circumferential surface of the first fastening part, a second fastening part may be provided on a rear portion of the cap, and a second screw thread may be formed on an inner circumferential surface of the second fastening part so as to be thread-coupled to the first screw thread.

A terminal insertion groove may be formed in a rear portion of the conductor, and the connection terminal may be inserted and coupled into the terminal insertion groove from a rear portion of the body.

The connection terminal may be disposed at an outer diameter side of the body so as to be orthogonal to the conductor, and one end of the connection terminal may be in contact with a rear end of the conductor.

The medical punching/cauterizing device according to the exemplary embodiment of the present invention may further include a fixing chuck interposed between the body and the cap and configured to fix the guide wire.

The fixing chuck may have therein a second through hole into which the guide wire is inserted, and the fixing chuck may have a cut-out portion formed in a longitudinal direction.

A second guide surface may be provided at a front end of the fixing chuck and disposed around the second through hole to guide the insertion of the guide wire, and a diameter of the second guide surface may decrease toward a rear portion of the fixing chuck.

A first inclined surface may be formed on an inner surface of a front portion of the cap, a diameter of the first inclined surface may decrease toward the front portion of the cap, a second inclined surface may be formed on an outer surface of a front portion of the fixing chuck so as to face the first inclined surface of the cap, and a diameter of the second inclined surface may decrease forward.

The guide wire may include: a punching/cauterizing part configured to puncture or cauterize a lesion part; and an extension part extending from the punching/cauterizing part, inserted into the handle, and electrically connected to the conductor.

A wire insertion groove may be formed at a center of the conductor so that the extension part of the guide wire is inserted into the wire insertion groove.

An insulation member may be provided on an outer surface of the extension part exposed to the outside of the handle.

The punching/cauterizing part of the guide wire may be formed to have any one of a straight shape, a hook shape, a loop shape, a triangular shape, and a diamond shape.

The medical punching/cauterizing device according to the exemplary embodiment of the present invention has, in one module, all the functions of a puncture needle, a guide wire, and an electrocautery the related art. That is, the medical punching/cauterizing device according to the exemplary embodiment of the present invention is a device capable of inserting the guide wire into a lesion part and simultaneously expanding a tract by performing electrocauterization, thereby improving a success rate of the procedure, reducing the number of procedures and the procedure time, and thus minimizing the complication associated with the procedure.

### DESCRIPTION OF DRAWINGS

FIG. 1 is a perspective view illustrating a medical punching/cauterizing device according to a first exemplary embodiment of the present invention.

FIG. 2 is a cross-sectional view illustrating the medical punching/cauterizing device according to the first exemplary embodiment of the present invention.

FIG. 3 is a detailed view of part A in FIG. 2.

FIG. 4 is a detailed view of part B in FIG. 2.

FIG. 5 is a perspective view illustrating a fixing chuck illustrated in FIG. 2.

FIG. 6 is a perspective view illustrating a medical punching/cauterizing device according to a second exemplary embodiment of the present invention.

FIG. 7 is a cross-sectional view illustrating the medical punching/cauterizing device according to the second exemplary embodiment of the present invention.

FIG. 8 is a detailed view of part C in FIG. 7.

FIGS. 9 to 12 are schematic views illustrating guide wires to be applied to the exemplary embodiments of the present invention.

### DETAILED DESCRIPTION

Hereinafter, exemplary embodiments of the present invention will be described with reference to the accompanying drawings, and the terms ‘front side’, ‘front portion’, ‘rear side, and ‘rear portion’ are used to clarify the subject matter of the invention. The front side or the front portion is defined as a side or a portion close to a position at which a guide wire is positioned, and the rear side or the rear portion is opposite to the front side or the front portion and defined as a side or a portion close to a position at which a handle is positioned.

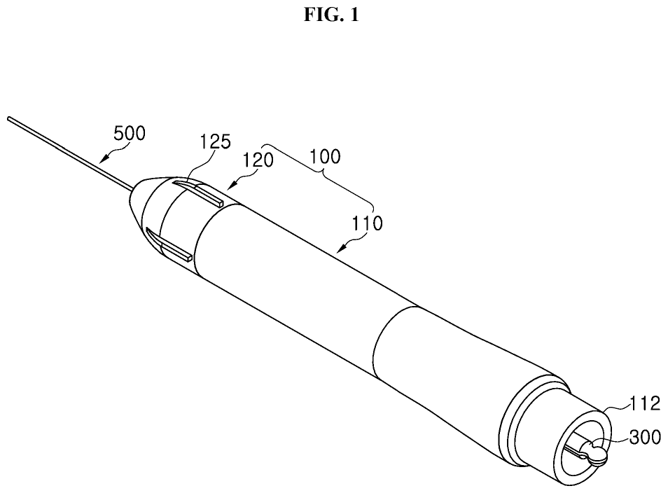

FIGS. 1 to 5 illustrate a medical punching/cauterizing device according to a first exemplary embodiment of the present invention. Referring to FIGS. 1 to 5, the medical punching/cauterizing device according to the first exemplary embodiment includes a handle 100, a fixing chuck 200, a connection terminal 300, a conductor 400, and a guide wire 500.

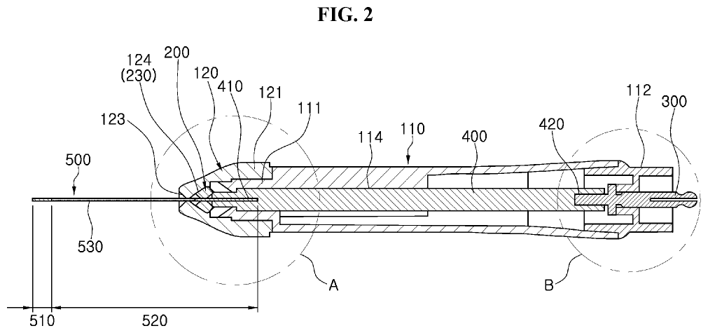

As illustrated in FIGS. 1 and 2, the handle 100 includes a body 110 configured to be substantially grasped by a user, and a cap 120 detachably coupled to a front portion of the body 110.

The body 110 may have an approximately cylindrical external shape. However, the shape of the body 110 is not limited as long as the body 110 has an external shape to be easily grasped by the user.

A first fastening part 111 may protrude forward from the front portion of the body 110 so that the cap 120 is fastened to the body 110. A first screw thread may be formed on an outer circumferential surface of the first fastening part 111. A conductor receiving groove 114 is formed in the body 110 so that the conductor 400 is received in the conductor receiving groove 114. The conductor receiving groove 114 may be formed to penetrate the body 110 from the front portion to a rear portion of the body 110. A power connection unit 112 is provided on the rear portion of the body 110, and a connector or the like of a power supply means (not illustrated) is connected to the power connection unit 112. The power connection unit 112 may be configured as a hollow body having a vacant internal space, and the connection terminal 300 is disposed in the internal space of the power connection unit 112 so as to be partially exposed to the outside.

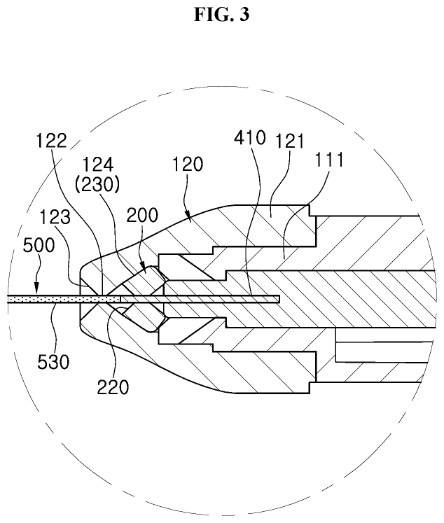

As illustrated in FIGS. 2 and 3, a first through hole 122 is formed in a longitudinal direction and disposed at a center of the cap 120 so that the guide wire 500 is inserted into the first through hole 122. A first guide surface 123 may be inclinedly formed on a front portion of the cap 120 and disposed around the first through hole 122. The first guide surface 123 guides the insertion of the guide wire 500 into the handle 100. Therefore, the first guide surface 123 may be tapered so that a diameter of the first guide surface 123 decreases toward a rear portion of the cap 120.

A second fastening part 121 may be provided on the rear portion of the cap 120 and coupled to the first fastening part 111 of the body 110. An inner diameter of the second fastening part 121 may correspond to an outer diameter of the first fastening part 111. A second screw thread may be formed on an inner circumferential surface of the second fastening part 121. Therefore, the first fastening part 111 of the body 110 and the second fastening part 121 of the cap 120 may be thread-coupled to each other.

A first inclined surface 124 may be formed on an inner surface of the front portion of the cap 120. The first inclined surface 124 may be tapered and inclined so that a diameter of the first inclined surface 124 decreases toward the front portion of the cap 120. The first inclined surface 124 is disposed to face a second inclined surface 230 of the fixing chuck 200 which will be described below. The first inclined surface 124 may press the second inclined surface 230 when the cap 120 is fastened to the body 110.

A rib 125 may be provided on an outer surface of the cap 120 in order to make it easy for the user to grasp the cap 120 when assembling or disassembling the body 110 and the cap 120. A plurality of ribs 125 may be provided radially at predetermined intervals, and each of the ribs 125 may be elongated in the longitudinal direction of the cap 120.

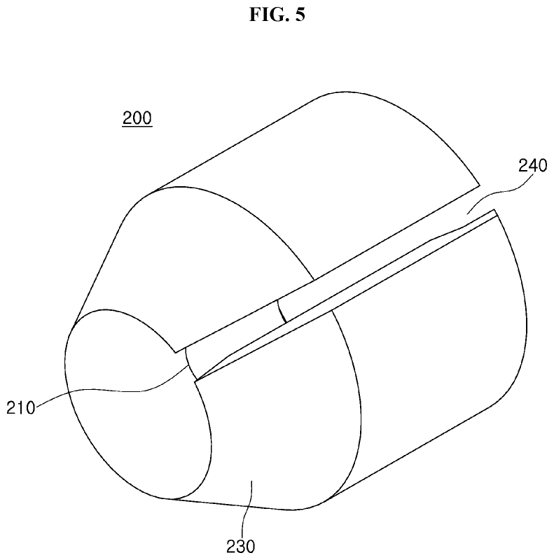

As illustrated in FIG. 5, the fixing chuck 200 is provided on the front portion of the body 110 and fixes the position of the guide wire 500. The fixing chuck 200 is configured as an approximately cylindrical hollow body. A cut-out portion 240 may be provided at one side of a circumference of the fixing chuck 200 and made by cutting the fixing chuck 200 in the longitudinal direction from a front end to a rear end of the fixing chuck 200.

A second through hole 210 is formed at a center of the fixing chuck 200 so that the guide wire 500 is inserted into the second through hole 210. A second guide surface 220 may be formed on a front portion of the fixing chuck 200 and disposed around the second through hole 210, and the second guide surface 220 may guide the insertion of the guide wire 500. The second guide surface 220 may be tapered so that a diameter of the second guide surface 220 decreases toward the center of the fixing chuck 200 so that the guide wire 500 may be easily inserted into the second through hole 210.

An outer diameter of the fixing chuck 200 may be tapered so that a diameter of the fixing chuck 200 decreases forward. That is, the second inclined surface 230 may be formed on the outer surface of the tip of the fixing chuck 200, and a diameter of the second inclined surface 230 may decrease forward. The second inclined surface 230 is disposed to face the first inclined surface 124 of the cap 120 and pressed by the first inclined surface 124 when the cap 120 is fastened to the body 110. When the second inclined surface 230 is pressed by the first inclined surface 124, the cut-out portion 240 of the fixing chuck 200 is closed, such that the fixing chuck 200 may securely fix the guide wire 500.

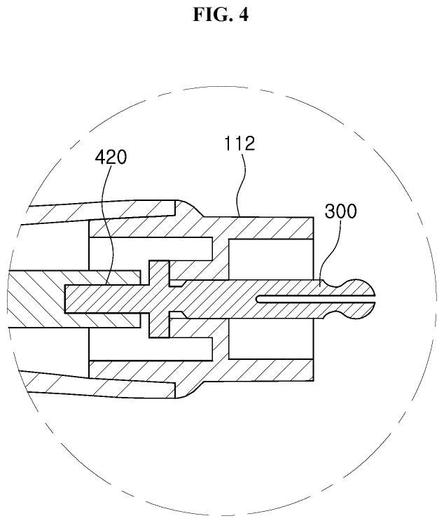

As illustrated in FIGS. 2 and 4, the connection terminal 300 is connected to the connector or the like of the power supply means and provides power to the guide wire 500. The connection terminal 300 is inserted and coupled into the terminal receiving groove 115 formed in the rear portion of the body 110 and disposed in an axial direction of the handle 100. Therefore, the axial direction of the connection terminal 300 and the axial direction of the handle 100 are coincident with each other.

For example, the power supply means may be a high-frequency oscillator, and the connection terminal 300 may provide the guide wire 500, through the conductor 400, with high-frequency waves generated by the high-frequency oscillator. One end of the connection terminal 300 may be connected to a rear end of the conductor 400, and the other end of the connection terminal 300 may be exposed to the outside of the body 110 so as to be connected to the high-frequency oscillator.

The connection terminal 300 needs to be disposed at a position at which the connection terminal 300 does not interfere with the user when the user grasps the handle 100. Therefore, the connection terminal 300 may be disposed to be inserted in the direction from the rear portion to the front portion of the body 110. That is, the connection terminal 300 and the conductor 400 may be disposed in a straight line, and a front end of the connection terminal 300 may be in contact with the rear end of the conductor 400, such that the connection terminal 300 and the conductor 400 may be electrically connected to each other.

As illustrated in FIGS. 2 to 4, the conductor 400 may serve as a medium for electrically connecting the connection terminal 300 and the guide wire 500. The conductor 400 may be received in the conductor receiving groove 114 of the body 110 and formed to correspond to the shape of the conductor receiving groove 114. For example, the conductor receiving groove 114 may be formed to have a circular cross section, and the conductor 400 may be formed in a cylindrical shape.

The rear end of the conductor 400 is electrically connected to the connection terminal 300, and a front end of the conductor 400 is exposed from the front portion of the body 110.

A wire insertion groove 410 may be formed in a front portion of the conductor 400 and disposed in the longitudinal direction. A part of a rear portion of the guide wire 500 is inserted into the wire insertion groove 410, and the guide wire 500 receives power from the conductor 400.

A terminal insertion groove 420 may be formed in a rear portion of the conductor 400 and disposed in the longitudinal direction. One end of the connection terminal 300 is inserted into the terminal insertion groove 420. Therefore, the power provided from the power supply means may be transmitted in the order of the connection terminal 300, the conductor 400, and the guide wire 500.

Diameters of the guide wires 500 may be variously set, as necessary. For example, the guide wires 500 may have various diameters of 038″, 035″, 025″, 018″, 016″, 014″, 012″, and the like, and any one of the guide wires 500 may be mounted on the handle 100, as necessary.

The guide wire 500 may serve as a needle for puncturing a diseased part or serve to perform electrocauterization. Therefore, the guide wire 500 may be made of a conductive material, for example, metal such as stainless steel in order to enable the function of puncturing the diseased part or performing the electrocauterization. The guide wire 500 is electrically connected to the conductor 400 and supplied with high-frequency waves from the conductor 400 and an electrode connected to the power supply means, for example, the high-frequency oscillator.

As illustrated in FIG. 2, the guide wire 500 may include a punching/cauterizing part 510, and an extension part 520 extending rearward from the punching/cauterizing part 510.

The punching/cauterizing part 510 punctures a diseased part or performs electrocauterization for stopping bleeding from the diseased part. The punching/cauterizing part 510 is provided at a front end of the guide wire 500 and may have a length of 3 to 5 mm. The punching/cauterizing part 510 may be tapered so that a diameter of the punching/cauterizing part 510 gradually decreases forward.

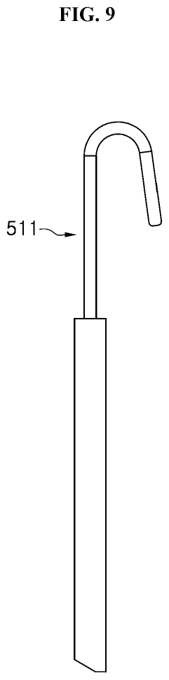

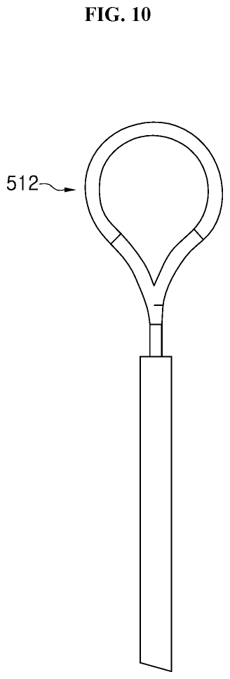

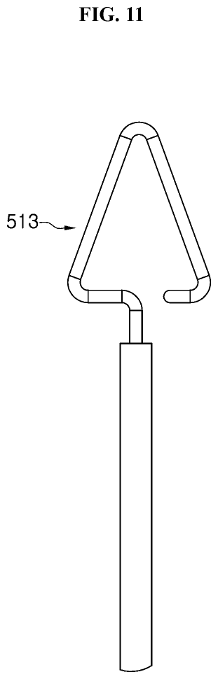

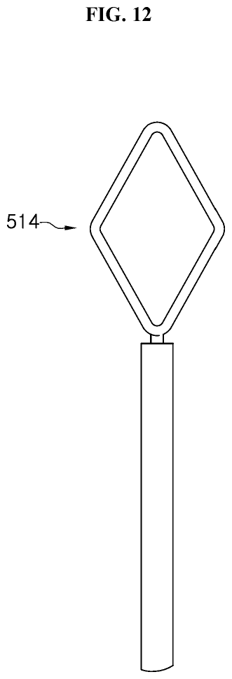

The punching/cauterizing part 510 may be formed integrally with a front portion of the extension part 520, or the punching/cauterizing part 510 may be manufactured separately from the extension part 520 and then coupled to the front portion of the extension part 520. In the first exemplary embodiment, the punching/cauterizing part 510 may be formed straight. However, as illustrated in FIGS. 9 to 12, the punching/cauterizing part 510 may have a hook shape 511, a loop shape 512, a triangular shape 513, a diamond shape 514, or the like, as necessary.

A rear end of the extension part 520 is inserted into the handle 100 and electrically connected to the conductor 400. For example, the insertion groove is formed at the center of the conductor 400, and the extension part 520 is inserted into the insertion groove, such that the extension part 520 may be supplied with high-frequency waves from the conductor 400.

An insulation member 530 may be provided on an outer portion of the extension part 520. The insulation member 530 may prevent the electrocauterization from being performed on other body tissue instead of a lesion part when the high-frequency waves are transmitted to the guide wire 500. That is, the insulation member 530 may be provided on a part of the extension part 520 exposed to the outside of the handle 100, except for the punching/cauterizing part 510 disposed at the tip of the guide wire 500 and configured to perform the electrocauterization. The insulation member 530 may be formed by applying a material such as polytetrafluoroethylene (PTFE) onto an outer surface of the extension part 520.

Meanwhile, lumens such as blood vessels or an esophagus may be narrowed due to diseases occurring in the human body and thus functions of the lumens may deteriorate, or in severe cases, lumens may perform no functions. For example, examples of the narrowing of the lumen may include a case in which an esophagus is narrowed due to esophageal cancer, a case in which a smooth blood circulation is not achieved due to arteriosclerosis, or a case in which a tract through which the bile may flow from the liver is narrowed.

Because food, blood, or bile cannot flow smoothly in the state in which the lumen is narrowed as described above, it is necessary to ensure a tract by expanding the narrowed lumen. In this case, as a method of expanding the narrowed lumen and ensuring the tract, an automatically inflatable medical device (hereinafter, referred to as a ‘stent’) is inserted into the lumen, as an example.

The stent generally has a cylindrical structure as a whole and has elasticity, such that the stent may be contracted when external force is applied to the stent, and the stent autonomously expands and returns to an original shape when the external force is eliminated.

The medical punching/cauterizing device according to the exemplary embodiment of the present invention may be used for stent implantation using endoscopic ultrasound (EUS). That is, when performing ultrasonic endoscopic cirrhotic stent drainage, the guide wire 500 may be connected to a stent insertion tool without a separate puncture needle, the guide wire 500 may be used to attempt to directly puncture a lesion, part and then the stent may be inserted.

As described above, the medical punching/cauterizing device according to the exemplary embodiment of the present invention may be inserted into the lumen in the state in which the stent, which is used to expand the narrowed lumen such as a blood vessel or an esophagus, is mounted on an outer portion of the guide wire. Therefore, as necessary, the process of implanting the stent may be continuously performed after the cauterization using the guide wire.

Further, the medical punching/cauterizing device according to the exemplary embodiment of the present invention may be used to stop bleeding from the bleeding site by performing the electrocauterization using the guide wire 500 on the bleeding site when arteriovenous hemorrhage occurs. That is, it is possible to prevent hemorrhage by introducing a guide catheter into the bleeding site, inserting the straight guide wire 500 into the guide catheter, connecting the power supply means to the guide wire 500, and cauterizing a wall of a blood vessel in the bleeding site to block a punctured portion or induce thrombus in the punctured portion.

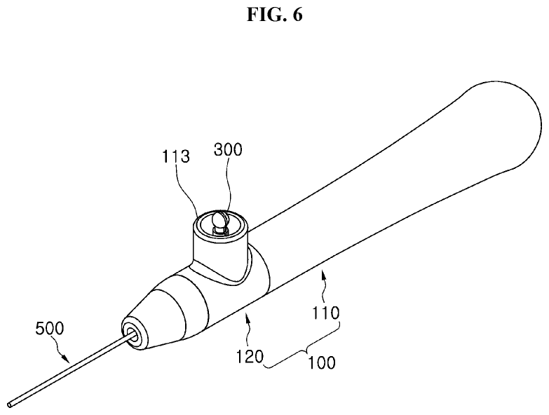

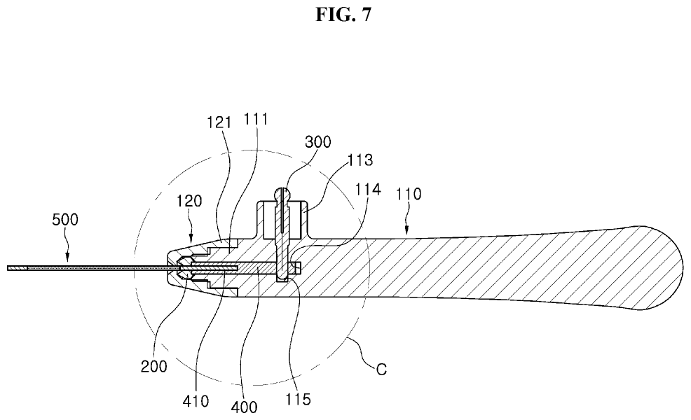

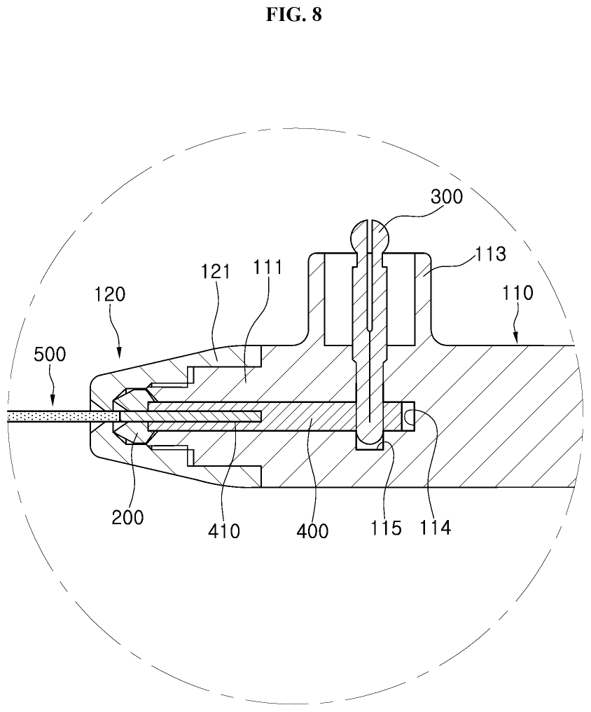

FIGS. 6 to 8 illustrate a medical punching/cauterizing device according to a second exemplary embodiment of the present invention. Referring to FIGS. 6 to 8, the medical punching/cauterizing device according to the second exemplary embodiment is similar to the medical punching/cauterizing device according to the first exemplary embodiment, except for some components of the handle 100. Therefore, detailed descriptions of other components, except for the handle 100, will be omitted.

The handle of the medical punching/cauterizing device according to the second exemplary embodiment is configured such that the axial direction of the connection terminal 300 and the axial direction of the handle are orthogonal to each other. That is, the handle includes the body 110 and the cap 120, and a terminal receiving groove 115 is formed in the body 110 and disposed in a direction from an outer diameter to a center of the body 110. In this case, the terminal receiving groove 115 may be formed to intersect the conductor receiving groove 114 of the body 110. Therefore, the conductor 400, which is received in the conductor receiving groove 114, and the connection terminal 300, which is disposed in the terminal receiving groove 115, are orthogonal to each other and in contact with each other.

In addition, since the terminal receiving groove 115 is formed at the outer diameter side of the body 110, a power connection unit 113 is also provided at the outer diameter side of the body 110.

In the second exemplary embodiment, the conductor 400 is installed to be inserted into a part of the front portion of the body 110 without penetrating the body 110 from one end to the other end of the body 110. Therefore, a length of the conductor 400 may be shorter in the medical punching/cauterizing device according to the second exemplary embodiment than in the medical punching/cauterizing device according to the first exemplary embodiment.

What is claimed is:

1. A medical punching/cauterizing device comprising:
a handle having a body, and a cap detachably coupled to a front portion of the body;
a connection terminal coupled to the body and electrically connected to a power supply means;
a conductor provided in the front portion of the body and having one end electrically connected to the connection terminal; and
a guide wire provided on a front portion of the handle and having one end electrically connected to the conductor.

2. The medical punching/cauterizing device of claim 1, wherein the cap has a first through hole into which the guide wire is inserted, a first guide surface is formed on a front portion of the cap and disposed around the first through hole, and a diameter of the first guide surface decreases toward a rear portion of the cap.

3. The medical punching/cauterizing device of claim 1, wherein a first fastening part is provided on the front portion of the body, a first screw thread is formed on an outer circumferential surface of the first fastening part, a second fastening part is provided on a rear portion of the cap, and a second screw thread is formed on an inner circumferential surface of the second fastening part so as to be thread-coupled to the first screw thread.

4. The medical punching/cauterizing device of claim 1, wherein a terminal insertion groove is formed in a rear portion of the conductor, and the connection terminal is inserted and coupled into the terminal insertion groove from a rear portion of the body.

5. The medical punching/cauterizing device of claim 1, wherein the connection terminal is disposed at an outer diameter side of the body so as to be orthogonal to the conductor, and one end of the connection terminal is in contact with a rear end of the conductor.

6. The medical punching/cauterizing device of claim 1, further comprising:
a fixing chuck interposed between the body and the cap and configured to fix the guide wire.

7. The medical punching/cauterizing device of claim 6, wherein the fixing chuck has therein a second through hole into which the guide wire is inserted, and the fixing chuck has a cut-out portion formed in a longitudinal direction.

8. The medical punching/cauterizing device of claim 7, wherein a second guide surface is provided at a front end of the fixing chuck and disposed around the second through hole to guide the guide wire to be inserted into the second through hole, and a diameter of the second guide surface decreases toward a rear portion of the fixing chuck.

9. The medical punching/cauterizing device of claim 6, wherein a first inclined surface is formed on an inner surface of a front portion of the cap, a diameter of the first inclined surface decreases toward the front portion of the cap, a second inclined surface is formed on an outer surface of a front portion of the fixing chuck so as to face the first inclined surface of the cap, and a diameter of the second inclined surface decreases forward.

10. The medical punching/cauterizing device of claim 1, wherein the guide wire comprises:
a punching/cauterizing part configured to puncture or cauterize a lesion part; and
an extension part extending from the punching/cauterizing part, inserted into the handle, and electrically connected to the conductor.

11. The medical punching/cauterizing device of claim 10, wherein a wire insertion groove is formed at a center of the conductor so that the extension part of the guide wire is inserted into the wire insertion groove.

12. The medical punching/cauterizing device of claim 10, wherein an insulation member is provided on an outer surface of the extension part exposed to the outside of the handle.

13. The medical punching/cauterizing device of claim 10, wherein the punching/cauterizing part of the guide wire is formed to have any one of a straight shape, a hook shape, a loop shape, a triangular shape, and a diamond shape.
